(19) United States
(12) Patent Application Publication
(10) Pub. No.: US 2025/0318860 A1
(43) Pub. Date: Oct. 16, 2025

(54) IMPLANTABLE NON-FUSION GROWTH MODULATING SYSTEM

(71) Applicant: Paul E. Kraemer, Westfield, IN (US)

(72) Inventor: Paul E. Kraemer, Westfield, IN (US)

(21) Appl. No.: 19/205,006

(22) Filed: May 12, 2025

Related U.S. Application Data
(63) Continuation-in-part of application No. 19/040,342, filed on Jan. 29, 2025.
(60) Provisional application No. 63/626,183, filed on Jan. 29, 2024.
(60) Provisional application No. 63/648,286, filed on May 16, 2024.

(51) Int. Cl. A61B 17/70; A61B 17/00

(52) U.S. Cl. CPC A61B 17/70; A61B 2017/00876

(57) ABSTRACT

An implantable non-fusion growth modulating system includes a series of vertebral body devices rigidly attached to an anterior column of a spine. Each of the series of vertebral body devices includes a fastening mechanism that attaches the vertebral body device to the anterior column and a magnetic or electromagnetic device, wherein opposing magnetic force from same-pole magnets provide counteracting force to an underlying deforming force of the spine. The series of vertebral body devices are not attached to one another through a connecting device. The magnetic or electromagnetic device may be a solid core magnet or multiple electromagnetic cores within a non-magnetic device. A power source and associated electrical circuitry may be provided within the system when the magnetic or electromagnetic device is an electromagnetic device.

### CROSS-REFERENCE TO RELATED APPLICATION

This application is a continuation-in-part of U.S. application Ser. No. 19/040,342, filed 29 Jan. 2025 (“the '342 application”), now pending, which claims the benefit of U.S. provisional application No. 63/626,183, filed 29 Jan. 2024 (“the '183 provisional”). This application also claims the benefit of U.S. provisional application No. 63/648,286, filed 16 May 2024 (“the '286 provisional”). The '342 application, the '183 provisional, and the '286 provisional are hereby incorporated by reference as though fully set forth herein.

### FIELD OF THE DISCLOSURE

The present disclosure generally relates to scoliosis correction, and more particularly, to an implantable non-fusion growth modulating system that may control growth and counteract deforming forces in the immature or young/flexible spine.

### BACKGROUND

Adolescent and juvenile scoliosis is a common and potentially debilitating condition that often requires substantial medical care, including multilevel fusion, at a young age. Despite extensive research, a causal link to genetics, the environment, or other factors has not been made. Treatment is available but comes at a risk as well as loss of mobility, and many patients are left with sometimes suboptimal options, especially for small to moderate curves. Multilevel posterior fusion is the standard for substantial curves. Non-fusion alternatives include bracing and tether systems that may be subject to mechanical failure and may be effective only in narrow populations.

### SUMMARY

Embodiments of the present disclosure may provide an implantable non-fusion growth modulating system comprising: a series of vertebral body devices rigidly attached to an anterior column of a spine, each of the series of vertebral body devices comprising: a fastening mechanism that attaches the vertebral body device to the anterior column; and a magnetic or electromagnetic device, wherein opposing magnetic force from same-pole magnets provide counteracting force to an underlying deforming force of the spine, wherein the series of vertebral body devices are not attached to one another through a connecting device. The series of vertebral body devices may be placed at or near apices of a deformity of the spine. The connecting device may be a rod, a plate, or a tether. The fastening mechanism may be a bolt or a screw. The fastening mechanism may be placed open or may be placed through a mini-open approach. The fastening mechanism may be amenable to robotic or navigation assistance. The magnetic or electromagnetic device may be secondarily attached to the fastening mechanism to repel magnetic or electromagnetic devices in adjacent vertebral body devices. The magnetic or electromagnetic device may not be secondarily attached to the fastening mechanism. The system may include a power source and associated electrical circuitry when the magnetic or electromagnetic device is an electromagnetic device. The magnetic or electromagnetic device may be a solid core magnet or multiple electromagnetic cores within a non-magnetic device. The non-magnetic device may be attached to a fixation device with each of the multiple electromagnetic cores controlled by its own power source. The magnetic or electromagnetic device may have multiple poles.

Other embodiments of the present disclosure may provide an implantable non-fusion growth modulating system comprising: a series of vertebral body devices rigidly attached to an anterior column of a spine, each of the series of vertebral body devices comprising: a fastening mechanism that attaches the vertebral body device to the anterior column; and a magnetic or electromagnetic device secondarily attached to the fastening mechanism, wherein opposing magnetic force from same-pole magnets provide counteracting force to an underlying deforming force of the spine, wherein the series of vertebral body devices are not attached to one another through a connecting device. The magnetic or electromagnetic device may be a solid core magnet or multiple electromagnetic cores within a non-magnetic device. The non-magnetic device may be attached to a fixation device with each of the multiple electromagnetic cores controlled by its own power source. The magnetic or electromagnetic device may have multiple poles.

Additional embodiments of the present disclosure may provide an implantable non-fusion growth modulating system comprising: a series of vertebral body devices rigidly attached to an anterior column of a spine, each of the series of vertebral body devices comprising: a fastening mechanism that attaches the vertebral body device to the anterior column; and a magnetic or electromagnetic device, wherein opposing magnetic force from same-pole magnets provide counteracting force to an underlying deforming force of the spine; and a power source and associated electrical circuitry when the magnetic or electromagnetic device is an electromagnetic device, wherein the series of vertebral body devices are not attached to one another through a connecting device.

### BRIEF DESCRIPTION OF THE DRAWINGS

For a more complete understanding of this disclosure, reference is now made to the following description, taken in conjunction with the accompanying drawings, in which:

FIG. 1 depicts an implantable non-fusion growth modulating system according to an embodiment of the present disclosure;

FIG. 2 depicts a three-dimensional view of static ligamentous convexity and how magnets may be placed relative to apical vertebrae;

FIG. 3 is a lateral schematic view of variable force magnetic cores;

FIG. 4 depicts multiple electromagnets within non-magnetic mounts; and

FIG. 5 depicts a posterior approach to embedding instrumentation according to aspects of the instant disclosure.

### DETAILED DESCRIPTION

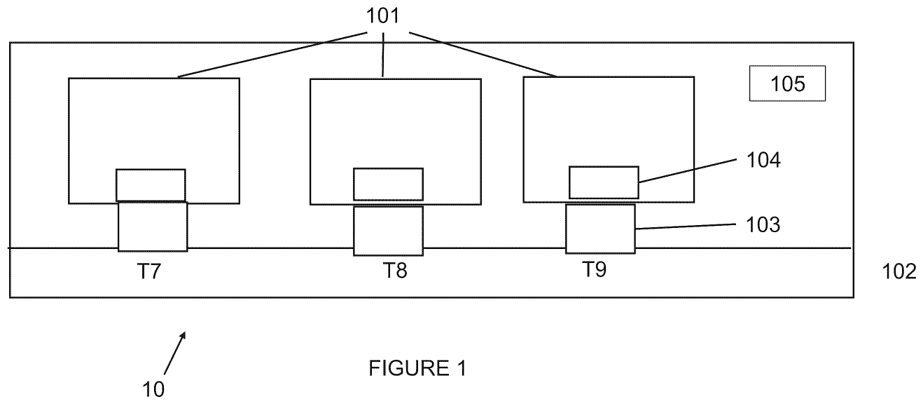

Embodiments of the present disclosure may provide an implantable non-fusion growth modulating system to control growth and counteract deforming forces in the immature or young/flexible spine. FIG. 1 schematically depicts an implantable non-fusion growth modulating system 10 according to an embodiment of the present disclosure. As depicted herein, a series of vertebral body devices 101, which may be magnets or electromagnets, may be rigidly attached to the anterior column of the spine 102 in a manner where opposing magnetic forces from same-pole magnets may provide counteracting force to the underlying deforming force of the diseased spine. The vertebral body devices 101 may be placed one on each of the treated vertebrae at or near the apices of the deformity. Unlike extant instrumentation, the vertebral body devices 101 are not physically attached to one another by any connecting device, such as a rod, plate, or tether. Accordingly, the vertebral body devices 101 will not restrict ongoing correction of the spine 102 over time. Further, they are not subject to mechanical failure due to a connecting device.

Each vertebral body device 101 may include a fastening mechanism 103, including, but not limited to, a bolt or screw, that may be attached rigidly to the anterior column of spine 102. The fastening mechanism 103 may be placed open or through a mini-open approach in embodiments of the present disclosure. It should be appreciated that the fastening mechanism 103 may be amenable to robotic or navigation assistance in embodiments of the present disclosure. There may be a “bolt-on” or single construct at each vertebral body in some embodiments of the present disclosure.

A magnetic or electromagnetic device 104 may be secondarily attached to the fastening mechanism 103 in a manner to repel adjacent devices within other vertical body devices 101 that also may be attached to the spine 102. Inclusion of the magnetic or electromagnetic device 104 within each vertical body device 101 may influence the conformation of the spine 102. The magnetic or electromagnetic device 104 may be secondarily attached to ensure accurate placement of the fastening mechanism 103 in embodiments of the present disclosure. However, it should be appreciated that it may not need to be secondarily attached in some embodiments of the present disclosure.

The implantable non-fusion growth modulating system also may include a power source and associated electrical conductors/circuitry 105 when an electromagnetic device may be used.

Either solid core magnet or multiple cores within a non-magnetic device may be employed within the implantable non-fusion growth modulating system in embodiments of the present disclosure. Multiple cores or poles of magnetism may allow separate forces in separate planes. More specifically, the T7 and T8 vertebrae may be repelled along the course of the device, but more strongly dorsally as opposed to ventral to produce some kyphosis. Multiple electromagnetic cores may be embedded within a plastic or plastic-like substance which may be attached to a fixation device, and each may be controlled by their own power source via separate conductors/circuitry in embodiments of the present disclosure. Multiple poles may be provided within the magnetic device such that separate current or magnets may be stronger or weaker along the magnetic device in some embodiments of the present disclosure. This may allow coronal and sagittal control independently.

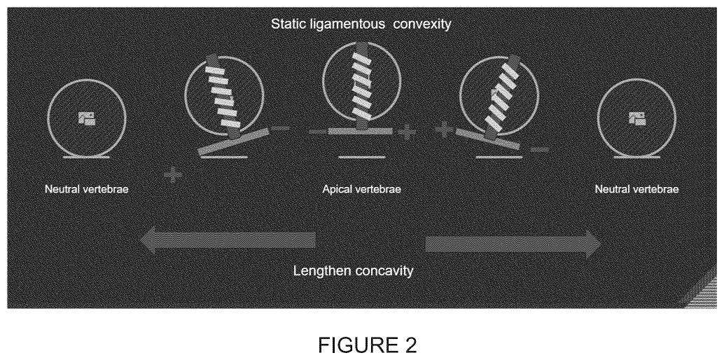

FIG. 2 depicts a three-dimensional view of static ligamentous convexity and how magnets may be placed relative to apical vertebrae. As this is a three-dimensional view, pure coronal curve is not fully depicted. Accordingly, the placement of the magnetic devices may be altered, including multiple poles within a device, in some embodiments of the present disclosure.

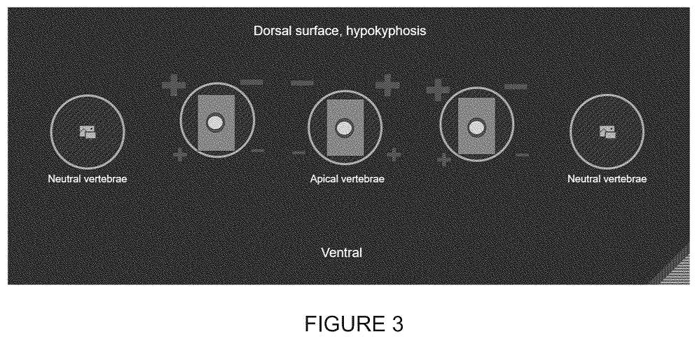

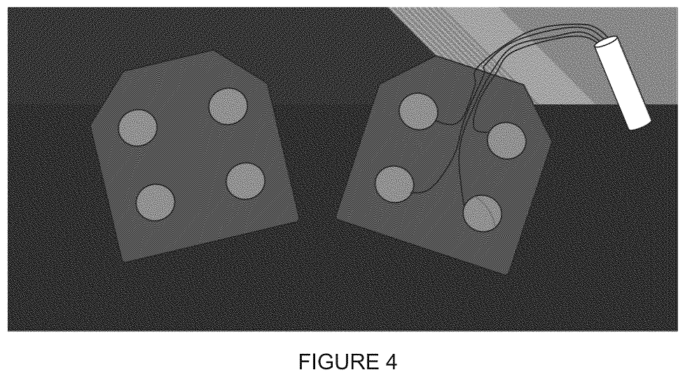

FIG. 3 is a lateral schematic view of variable force magnetic cores. FIG. 4 depicts multiple electromagnets within non-magnetic mounts. As depicted herein, electromagnetic cores may be within plastic mounts which may be individually controllable via a separately implantable battery/controller. This may be similar to a spinal cord stimulator.

The implantable non-fusion growth modulating system according to embodiments of the present disclosure employs magnetic forces rather than mechanical forces and therefore is not subject to fatigue failure. Further, as the vertebral body devices are not attached to one another, the degree of correction is not restricted. Moreover, electromagnetic configuration forces can be increased or decreased to dial in correction. In addition, magnetic repulsion becomes weaker at greater distances as correction is obtained. Accordingly, overcorrection may be modulated. Further, the system according to embodiments of the present disclosure may be employed through minimally invasive surgery and does not preclude spinal fusion.

Although the present disclosure and its advantages have been described in detail, it should be understood that various changes, substitutions and alterations can be made herein without departing from the spirit and scope of the disclosure as defined by the appended claims.

For example, it is contemplated that devices 101 as described herein may be placed using an anterior approach, such as via thoracotomy/thoracoscopy, and bolted on to the lateral aspect of a vertebral body (e.g., using a “squared coin”-based configuration).

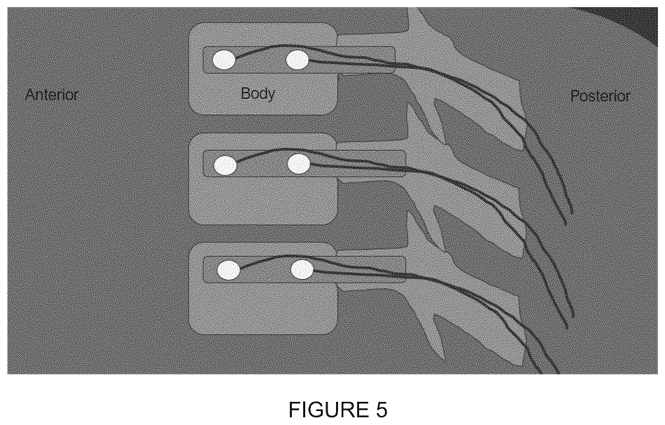

It is also contemplated, however, that devices 101 may be embedded within the anterior column, such as via a posterior pedicle screw-type trajectory, as shown in FIG. 5. Each device 101 for posterior placement (e.g., via pedicle) may include a cylindrical shaft that incorporates one or more magnetic or electromagnetic elements (or cores). The shaft may be fitted with threads to facilitate securing device 101 to the spinal column.

Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. An implantable non-fusion growth modulating system comprising:
a series of vertebral body devices rigidly attached to an anterior column of a spine, each of the series of vertebral body devices comprising:
a fastening mechanism that attaches the vertebral body device to the anterior column; and
a magnetic or electromagnetic device, wherein opposing magnetic force from same-pole magnets provide counteracting force to an underlying deforming force of the spine,
wherein the series of vertebral body devices are not attached to one another through a connecting device.

2. The system of claim 1, wherein the series of vertebral body devices are placed at or near apices of a deformity of the spine.

3. The system of claim 1, wherein the connecting device is a rod, a plate, or a tether.

4. The system of claim 1, wherein the fastening mechanism is a bolt or a screw.

5. The system of claim 1, wherein the fastening mechanism is placed open.

6. The system of claim 1, wherein the fastening mechanism is placed through a mini-open approach.

7. The system of claim 1, wherein the fastening mechanism is amenable to robotic or navigation assistance.

8. The system of claim 1, wherein the magnetic or electromagnetic device is secondarily attached to the fastening mechanism to repel magnetic or electromagnetic devices in adjacent vertebral body devices.

9. The system of claim 1, wherein the magnetic or electromagnetic device is not secondarily attached to the fastening mechanism.

10. The system of claim 1 further comprising:
a power source and associated electrical circuitry when the magnetic or electromagnetic device is an electromagnetic device.

11. The system of claim 1, wherein the magnetic or electromagnetic device is a solid core magnet.

12. The system of claim 1, wherein the magnetic or electromagnetic device is multiple electromagnetic cores within a non-magnetic device.

13. The system of claim 12, wherein the non-magnetic device is attached to a fixation device with each of the multiple electromagnetic cores controlled by its own power source.

14. The system of claim 1, wherein the magnetic or electromagnetic device has multiple poles.

15. An implantable non-fusion growth modulating system comprising:
a series of vertebral body devices rigidly attached to an anterior column of a spine, each of the series of vertebral body devices comprising:
a fastening mechanism that attaches the vertebral body device to the anterior column; and
a magnetic or electromagnetic device secondarily attached to the fastening mechanism, wherein opposing magnetic force from same-pole magnets provide counteracting force to an underlying deforming force of the spine,
wherein the series of vertebral body devices are not attached to one another through a connecting device.

16. The system of claim 1, wherein the magnetic or electromagnetic device is a solid core magnet.

17. The system of claim 1, wherein the magnetic or electromagnetic device is multiple electromagnetic cores within a non-magnetic device.

18. The system of claim 17, wherein the non-magnetic device is attached to a fixation device with each of the multiple electromagnetic cores controlled by its own power source.

19. The system of claim 1, wherein the magnetic or electromagnetic device has multiple poles.

20. An implantable non-fusion growth modulating system comprising:
a series of vertebral body devices rigidly attached to an anterior column of a spine, each of the series of vertebral body devices comprising:
a fastening mechanism that attaches the vertebral body device to the anterior column; and
a magnetic or electromagnetic device, wherein opposing magnetic force from same-pole magnets provide counteracting force to an underlying deforming force of the spine; and
a power source and associated electrical circuitry when the magnetic or electromagnetic device is an electromagnetic device,
wherein the series of vertebral body devices are not attached to one another through a connecting device.
